(19) United States
(12) Patent Application Publication
(10) Pub. No.: US 2022/0305245 A1
(43) Pub. Date: Sep. 29, 2022

(54) MEDICAMENT CONTAINMENT DEVICES AND ASSOCIATED COMPOSITIONS

(71) Applicant: SaNOtize Research and Development Corp., Vancouver (CA)

(72) Inventors: Gilly Regev, Vancouver (CA); Christopher C. Miller, North Vancouver (CA)

(73) Assignee: SaNOtize Research and Development Corp., Vancouver (CA)

(21) Appl. No.: 17/618,831

(22) PCT Filed: Jun. 12, 2020
(86) PCT No.: PCT/IB2020/000473

§ 371 Date: Dec. 13, 2021

Related U.S. Application Data
(60) Provisional application No. 62/860,632, filed on Jun. 12, 2019.

(51) Int. Cl. A61M 35/00

(52) U.S. Cl. CPC A61M 35/30; A61M 2202/0275

(57) ABSTRACT

Medicament containment devices and systems as well as related methods of use for administering a medicament or therapeutic composition or agent to a subject are disclosed and described. In one embodiment, a containment device can include a containment reservoir configured to receive a medicament or therapeutic agent. The containment device can be configured to be secured to a treatment area of a subject and the containment reservoir can be loaded with the medicament or therapeutic agent for administration to the subject in a localized targeted treatment area.

### PRIORITY DATA

This application claims the benefit of U.S. Provisional Patent Application Ser. No. 62/860,632 filed on Jun. 12, 2019, which is incorporated herein by reference.

### BACKGROUND

Nitric Oxide (NO) is a small, unstable diatomic molecule. It measures about 115 picometers in its bond length, and is soluble in hydrophilic and hydrophobic environments. It has free radical like nature, a short half-life, and it is easily oxidized into nitrogen dioxide. Within the body, nitric oxide can be endogenously produced by nitric oxide synthase enzymes (NOS), and is known to be involved in many physiological and pathological processes. For example, a low level of NO in the blood encourages vasodilation to prevent ischemic damage, helps wound healing, and is an effective anti-pathogen (e.g. antimicrobial and antiviral agent). Conversely, a high level of NO in the blood leads to tissue toxicity and contributes to inflammatory conditions like septic shock, diabetes, and arthritis.

### BRIEF DESCRIPTION OF THE DRAWINGS

For a fuller understanding of the nature and advantage of the present invention, reference is being made to the following detailed description and in connection with the accompanying drawings, in which:

FIG. 1A illustrates a medicament containment device in accordance with an embodiment of the present disclosure;

FIG. 1B illustrates a cross-sectional view of a portion of a medicament containment device in accordance with an embodiment of the present disclosure;

FIG. 2A illustrates various examples of medicament containment devices of different shape or configuration in accordance with embodiments of the present disclosure;

FIG. 2B illustrates various views of an example medicament containment device in accordance with an embodiment of the present disclosure;

FIG. 3A illustrates various examples of a containment reservoir portion of different medicament containment devices in accordance with an embodiment of the present disclosure;

FIG. 3B illustrates various views of an example containment reservoir portion of a medicament containment device in accordance with an embodiment of the present disclosure;

FIG. 4 illustrates a method of manufacturing a medicament containment device in accordance with an embodiment of the present disclosure;

FIGS. 5A-5H illustrate various steps of applying a medicament containment device to a toe of a subject in accordance with an embodiment of the present disclosure;

FIG. 6 illustrates how a medicament containment device can be applied to a toe on a right foot vs. a toe on a left foot in accordance with an embodiment of the present disclosure;

FIG. 7A illustrates a double-barreled syringe and cork-screw type dispensing nozzle in accordance with an embodiment of the present disclosure;

FIG. 7B illustrates an expanded view of a double-barreled syringe in accordance with an embodiment of the present disclosure;

FIG. 8A is a graph showing that nitric oxide releasing solution (NORS) has a time and dose dependent antifungal effect against Trichophyton Rubrum mycelial growth. X represents the NORS dose required for complete fungicidal effect;

FIG. 8B is a graph showing that NORS at dose X rapidly kills Trichophyton Mentagrophtes mycelia within 10 minutes;

FIG. 9A is a graph showing positive results of placebo (n=4) and treatment (n=8) groups. Samples were taken before the study (baseline) and 3, 17 and 31 days after the first treatment. Fisher's test was used to test significance (**=p<0.01; *=p<0.05);

FIG. 9B is a graph of placebo (n=7) and treatment (n=13). CSSS was taken on Day 1, before intervention and Day 31 of the study. Standard error means are shown. Two-way ANOVA was used to test significance (***=p<0.001);

FIG. 10 is an image of the results from ex-vivo study from subject #2 demonstrating that nail fungus was cultured from toe nail sample (A) and successful fungicidal effect after exposure of nitric oxide releasing gel (NORG) NORG-80 for 8 hours, daily for 7 days (B). Note that the spots in container B are nail clippings without fungal growth after NORG exposure;

FIG. 11 is a graph of C. acnes exposure to 60 mM NORG. ˜10⁵ cfu/mL of C. acnes was exposed to 60 mM NORS. No bacteria detection was found after 3 minutes. n=3 (**=p<0.01, ***=p<0.001);

FIG. 12 is a graph of measured release of nitric oxide. 1 mL of 60 mM NORS and NORG, pH 3.5 were injected into the flow-over device connected to the chemiluminescence with a nitrogen gas flow-rate of 1 L/min. n=3; and

FIG. 13 is a graph of the concentration of S. aureus after exposure to 60 mM NORS and 60 mM NORG at pH 3.5. N=3.

### DESCRIPTION OF EMBODIMENTS

Although the following detailed description contains many specifics for the purpose of illustration, a person of ordinary skill in the art will appreciate that many variations and alterations to the following details can be made and are considered to be included herein. Accordingly, the following embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs.

As used in this written description, the singular forms “a,” “an” and “the” provide express support for plural referents unless the context clearly dictates otherwise. Thus, for example, reference to “a particle” includes a plurality of particles.

In this application, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term, like “comprising” or “including,” in this written description it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

Occurrences of the phrase “in one embodiment,” or “in one aspect,” herein do not necessarily all refer to the same embodiment or aspect.

As used herein, “subject” refers to a mammal that may benefit from nitric oxide therapy, such as nitric oxide administered via presentation or application of a nitric oxide releasing solution (NORS) or nitric oxide releasing gel (NORG). In one aspect the mammal may be a human.

As used herein, the terms “treat,” “treatment,” or “treating” when used in conjunction with the administration of NORS or NORG, including compositions and dosage forms thereof, refers to administration to subjects who are either asymptomatic or symptomatic. In other words, “treat,” “treatment,” or “treating” can be to reduce, ameliorate or eliminate symptoms associated with a condition present in a subject, or can be prophylactic, (i.e. to prevent or reduce the occurrence of the symptoms in a subject). Such prophylactic treatment can also be referred to as prevention of the condition.

As used herein, the terms “formulation” and “composition” are used interchangeably and refer to a mixture of two or more compounds, elements, or molecules. In some aspects the terms “formulation” and “composition” may be used to refer to a mixture of one or more active agents with a carrier or other excipients. Compositions can take nearly any physical state, including solid, liquid (i.e. solution), or gas. Furthermore, the term “dosage form” can include one or more formulation(s) or composition(s) provided in a format for administration to a subject. In one example, a composition can be a solution that releases nitric oxide.

As used herein “NORS” refers to a nitric oxide (NO) releasing solution, composition, or substance. In one embodiment, a NORS can be a nitric oxide releasing gel (NORG). In one aspect, NO released from NORS/NORG may be a gas.

As used herein the terms “therapeutic agent” and “active agent” and the like can be used interchangeably and refer to an agent that can have a beneficial or positive effect on a subject when administered to the subject in an appropriate or effective amount. In one aspect, NO can be a therapeutic agent. The terms “additional active agent,” “supplemental active agent,” “secondary active agent,” and the like can be used interchangeably and refer to a compound, molecule, or material other than nitric oxide that has physiologic activity when administered to a subject in an effective amount. Exemplary additional active agents can include without limitation, antimicrobial agents (e.g. antifungal agents, antiviral agents, antibacterial agents), antioxidants, vitamins, etc.

As used herein, an “effective amount” of an agent is an amount sufficient to accomplish a specified task or function desired of the agent. A “therapeutically effective amount” of a composition, drug, or agent refers to a non-toxic, but sufficient amount of the composition, drug, or agent, to achieve therapeutic results in treating or preventing a condition for which the composition, drug, or agent is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician, veterinarian, or other qualified medical personnel using evaluations known in the art, it is recognized that individual variation and response to treatments may make the achievement of therapeutic effects a somewhat subjective decision. The determination of an effective amount or therapeutically effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986).

As used herein, a “dosing regimen” or “regimen” such as “treatment dosing regimen,” or a “prophylactic dosing regimen” refers to how, when, how much, and for how long a dose of a composition can or should be administered to a subject in order to achieve an intended treatment or effect.

As used herein, the terms “release” and “release rate” are used interchangeably to refer to the discharge or liberation, or rate thereof, of a substance, including without limitation a therapeutic agent, such as NO, from the dosage form or composition containing the substance. In one example, a therapeutic agent may be released in vitro. In another aspect, a therapeutic agent may be released in vivo.

As used herein, “immediate release” or “instant release” can be used interchangeably and refer to immediate or near immediate (i.e. uninhibited or unrestricted) release of an agent or substance, including a therapeutic agent, such as NO, from a composition or formulation.

As used herein, the term “controlled release” refers to non-immediate release of an agent or substance, including a therapeutic agent, such as NO, from a composition or formulation. Examples of specific types of controlled release include without limitation, extended or sustained release and delayed release. Any number of control mechanisms or components can be used to create a controlled release effect, including formulation ingredients or constituents, formulation properties or states, such as pH, an environment in which the formulation is placed, or a combination of formulation ingredients and an environment in which the formulation is placed. In one example, extended release can include release of a therapeutic agent at a level that is sufficient to provide a therapeutic effect or treatment for a non-immediate specified or intended duration of time. Specific controlled release profiles can include sustained or extended release, pulsatile release, delayed release, etc.

As used herein, comparative terms such as “increased,” “decreased,” “better,” “worse,” “higher,” “lower,” “enhanced,” “maximized,” “minimized,” and the like refer to a property of a device, component, composition, or activity that is measurably different from other devices, components, compositions or activities that are in a surrounding or adjacent area, that are similarly situated, that are in a single device or composition or in multiple comparable devices or compositions, that are in a group or class, that are in multiple groups or classes, or as compared to the known state of the art. For example, an area of tissue that has been treated with nitric oxide therapy, such as a nail and/or surrounding nail tissue, can have an “improved” appearance or condition (e.g. by reduction of mycotic pathogens) after receiving the treatment as compared to its state prior to treatment.

The term “coupled,” as used herein, is defined as directly or indirectly connected in a chemical, mechanical, electrical or nonelectrical manner. “Directly coupled” objects or structures are in physical contact and are attached. Objects described herein as being “adjacent to” each other may be in physical contact with each other, in close proximity to each other, or in the same general region or area as each other, as appropriate for the context in which the phrase is used. Occurrences of the phrase “in one embodiment,” or “in one aspect,” herein do not necessarily all refer to the same embodiment or aspect.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments described herein are, for example, capable of operation in other orientations than those illustrated or otherwise described herein.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. Unless otherwise stated, use of the term “about” in accordance with a specific number or numerical range should also be understood to provide support for such numerical terms or range without the term “about”. For example, for the sake of convenience and brevity, a numerical range of “about 50 ml to about 80 ml” should also be understood to provide support for the range of “50 ml to 80 ml.” Furthermore, it is to be understood that in this specification support for actual numerical values is provided even when the term “about” is used therewith. For example, the recitation of “about” 30 should be construed as not only providing support for values a little above and a little below 30, but also for the actual numerical value of 30 as well.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually, and further including decimal or fraction values such as 1.8, 2.3, 3.7, and 4.2.

This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

Reference in this specification may be made to devices, structures, systems, or methods that provide “improved” performance. It is to be understood that unless otherwise stated, such “improvement” is a measure of a benefit obtained based on a comparison to devices, structures, systems or methods in the prior art. Furthermore, it is to be understood that the degree of improved performance may vary between disclosed embodiments and that no equality or consistency in the amount, degree, or realization of improved performance is to be assumed as universally applicable.

### Example Embodiments

An initial overview of invention embodiments is provided below and specific embodiments are then described in further detail. This initial summary is intended to aid readers in understanding the technological concepts more quickly, but is not intended to identify key or essential features thereof, nor is it intended to limit the scope of the claimed subject matter.

As described above, NO can provide a number of beneficial effects and is endogenously produced by nitric oxide synthase enzymes (NOS). For example, a low level of NO in the blood encourages vasodilation to prevent ischemic damage, helps wound healing, and is an effective antimicrobial agent. However, a high level of NO in the blood can lead to tissue toxicity and can contribute to inflammatory conditions like septic shock, diabetes, and arthritis. Thus, while NO can provide a variety of therapeutic effects, it can be desirable to employ various safeguards to minimize over-exposure to NO.

Outside of the body, NO can be produced through various methods. For example, NO can be produced by the catalyzed oxidation of ammonia, the extremely endothermic reaction of nitrogen and oxygen as free gases, and from acidified nitrites. Production of NO from acidified nitrites can be measured in at least 3 ways. For example, NO production can be measured using chemiluminescence, Gas Chromatography-Mass Spectrometry (GC-MS), and/or indirectly through the use of a Griess Reagent and spectrophotometer set to a wavelength of 543 nm. One example of specific NO quantification devices and methods can be found in Applicant's co-pending U.S. patent application Ser. No. 16/541,084, filed Aug. 14, 2019, which is incorporated herein by reference. Chemiluminescence is a well-known analytical technique that generally involves the emission of light as a result of a chemical reaction. More specifically, decay of a molecule in an excited state to a lower energy state can cause an emission of light, which can be detected by a chemiluminescence analyzer/detector. As one example, nitric oxide (NO) can react with ozone (O₃) to produce excited NO that subsequently decays to a lower energy state and emits electromagnetic radiation that is photoelectrically detectable.

An acidified nitrite solution can be based on the following reaction:

1. NO₂⁻+H⁺ [symbol] HNO₂

2a. 2HNO₂ [symbol] N₂O₃+H₂O [symbol] H₂O+NO+NO₂

2b. 3HNO₂ [symbol] 2NO+NO₃⁻+H₂O+H⁺

This can also be the basis for what can be called a Nitric Oxide Releasing Solution or Substance (NORS). A nitrite agent, like sodium or potassium nitrite, for example, can react with a proton donor. The proton donor can be any kind of acidifying agent to allow the reaction to proceed, but may add undesirable intermediates or by-products depending on which acidifying agent is used. Acidified nitrite solutions have antimicrobial properties and can be used to fight various types of infections including fungal infections caused by Tinea pedis (athlete's foot), bacterial infections from Propionibacterium acnes, and various viral infections, for example. The effectiveness of the antimicrobial activity can be dependent on a variety of factors, such as the pH of the composition, the concentration of nitrites available for the reaction, the concentration of acidifying agent to allow the reaction to proceed, and other stabilizing factors. The prospect of a NORS allows for the use of NO in treatments without having to use NO gas from a pressurized canister.

While a NORS and/or NORG can be a very effective antimicrobial composition, it can be challenging to maintain effective concentrations of NO at the treatment area while minimizing any toxic side-effects to the subject. Accordingly, the present disclosure is directed to medicament containment/administration devices and associated compositions that can help maintain effective concentrations of NO at the treatment area or situs while minimizing toxic side effects by substantially containing the NO within a containment reservoir (e.g. preventing or substantially preventing release out of the containment reservoir in any direction other than a direction toward an administration situs of a subject).

It is also noted that when discussing the medicament containment devices, NORS/NORG compositions, treatment systems, therapeutic systems, and methods described herein, these relative discussions can be considered applicable to the other examples, whether or not they are explicitly discussed in the context of that example. Thus, for example, in discussing a nitrite agent related to a therapeutic system, such disclosure is also relevant to and directly supported in the context of the treatment systems, medicament containment devices, NORS/NORG compositions, and methods described herein, and vice versa.

In further detail, a NORS can include a nitrite agent, an acidifying agent, and an aqueous vehicle. The NORS can be formulated to release from about 1 ppb NO to about 10,000 ppm NO over a period of from about 30 seconds to about 48 hours, or from about 10 minutes to about 24 hours. More specifically, the nitrite agent and the acidifying agent can be combined together in the aqueous carrier to form a nitric oxide releasing solution or substance (NORS) as an antimicrobial agent, such as an antibacterial agent, an antifungal agent, an antiviral agent, or a combination thereof, for example.

A variety of nitrite agents can be included in the NORS. Generally, any nitrite that can be acidified to generate nitric oxide can be used. Non-limiting examples can include sodium nitrite, potassium nitrite, barium nitrite, calcium nitrite, nitrite orotate, amyl nitrite, magnesium nitrite, the like, or a combination thereof.

The amount of nitrite agent can depend on the type of composition being employed. In some examples, the NORS can be in the form of a solution, a cream, a gel, an ointment, or the like. Depending on the particular formulation type, the nitrite agent can generally be present in the NORS in an amount from about 0.01 wt % to about 10 wt % based on the total weight of the composition. In other examples, the nitrite agent can be present in the NORS in an amount of from about 0.01 wt % to about 0.1 wt %, from about 0.1 wt % to about 1 wt %, from about 1 wt % to about 5 wt %, or from about 5 wt % to about 10 wt % based on a total weight of the NORS. In some additional examples, the nitrite agent can be present in the NORS in an amount of from about 0.1 millimolar (mM) to about 500 mM. In other examples, the nitrite agent can be present in the NORS in an amount of from about 0.1 mM to about 10 mM, from about 1 mM to about 10 mM, from about 5 mM to about 50 mM, from about 20 mM to about 200 mM, or from about 50 mM to about 500 mM. In some specific examples, the nitrite agent can be present in the NORS in an amount of from about 50 mM to about 150 mM.

A variety of acidifying agents can also be included in the NORS. Generally, any acidifying agent that is suitable to react with the nitrite agent without generating undesirable by-products can be used. In some specific examples, the acidifying agent can include an organic acid, such as ascorbic acid, ascorbyl palmitate, salicylic acid, malic acid, lactic acid, citric acid, formic acid, benzoic acid, tartaric acid, the like or a combination thereof. In some additional examples, the acidifying agent can include an inorganic acid, such as hydrochloric acid, sulfuric acid, phosphoric acid, the like, or a combination thereof. In some further examples, the acidifying agent can include a combination of an organic acid and an inorganic acid.

The acidifying agent can also be present in the NORS in a variety of amounts, depending on the form of the NORS. In some examples, the NORS can be in the form of a solution, a gel, a cream, an ointment, or the like. Depending on the formulation type, the acidifying agent can generally be included in the NORS in an amount to achieve a pH of from about 2.5 to about 5, or from about 3 to about 4. In some specific examples, the acidifying agent can be present in the NORS in an amount of from about 0.01 wt % to about 20 wt % based on a total weight of the NORS. In some specific examples, the acidifying agent can be included in the NORS in an amount of from about 0.01 wt % to about 0.1 wt %, from about 0.1 wt % to about 1 wt %, from about 1 wt % to about 5 wt %, from about 5 wt % to about 10 wt %, or from about 10 wt % to about 20 wt % based on a total weight of the NORS. In some additional examples, the acidifying agent can be present in the NORS in an amount of from about 0.1 millimolar (mM) to about 500 mM. In other examples, the acidifying agent can be present in the NORS in an amount of from about 0.1 mM to about 10 mM, from about 1 mM to about 10 mM, from about 5 mM to about 50 mM, from about 20 mM to about 200 mM, or from about 50 mM to about 500 mM. In some specific examples, the acidifying agent can be present in the NORS in an amount of from about 50 mM to about 150 mM.

Thus, the nitrite agent and the acidifying agent can be present in the NORS in a variety of amounts, depending on the particular type of formulation. In some examples, the nitrite agent and the acidifying agent can be present in the NORS at a weight ratio of from about 1:10 to about 10:1. In other examples, the nitrite agent and the acidifying agent can be present in the NORS at a weight ratio of from about 1:5 to about 5:1. In still other examples, the nitrite agent and the acidifying agent can be present in the NORS at a weight ratio of from about 1:3 to about 3:1, or from about 1:2 to about 2:1.

In some embodiments, the acidifying agent can be present in an excess amount over and above an amount needed to exhaust the available supply of nitrite in forming NO. In such cases, the surplus amount of acidifying agent can have a supplemental or additional therapeutic effect on the subject as a supplemental or additional active agent over and above a therapeutic effect provided by the NO. For example, when the acidifying agent is salicylic acid, any surplus or excess amount over the amount needed to react with nitrite and create NO, can have its own therapeutic effect on conditions such as tinea pedis and onychomycosis. Salicylic acid is known as an active ingredient in various medicaments formulated to treat skin conditions because of its desquamation action. For example, salicylic acid may be used to treat wars, acne, tinea pedis, onychomycosis, etc. Desquamation of dead skin cells can further enhance the effect that NO has on infected live tissue by clearing the area so that a higher NO concentration reaches the live infected tissue. Other acids besides salicylic acid may also have a desquamation effect and/or otherwise aid in penetration enhancement of NO. For example, salicylic or other acids may hydrate the keratin of finger or toe nails and thus render the nail more permeable to NO as well as provide greater access to the nail bed and other areas of infected tissue which are typically difficult to penetration and/or reach.

A variety of aqueous carriers can be included in the NORS. Non-limiting examples can include water, phosphate-buffered saline (PBS), Dulbecco's PBS, Alsever's solution, Tris-buffered saline (TBS), balanced salt solutions (BSS), such as Hank's BSS, Earle's BSS, Grey's BSS, Puck's BSS, Simm's BSS, Tyrode's BSS, BSS Plus, Ringer's lactate solution, normal saline (i.e. 0.9% saline), ½ normal saline, the like, or a combination thereof. Because the carrier can provide an environment in which the nitrite agent and the acidifying agent can react to produce nitric oxide, it can be beneficial to contain or house the NORS in a pressurized container to inhibit or minimize production of nitric oxide prior to application. After dispensing the NORS from the pressurized container, nitric oxide generation can proceed rapidly.

In other examples, one or more of the nitrite agent and the acidifying agent can be encapsulated. In some examples, the nitrite agent can be encapsulated. In some examples, the acidifying agent can be encapsulated. Where encapsulation is employed, the encapsulant material can typically be present at a weight ratio of from about 0.05:1 to 10:1 with the nitrite agent, the acidifying agent, or a combination thereof. Where one or more of the nitrite agent and the acidifying agent are encapsulated, the NORS can either be stored in a pressurized container as described above, the encapsulation material can be frangible upon dispensing, or the like to minimize NO production prior to application of the NORS. One example of an encapsulated NORS can be found in Applicant's co-pending U.S. patent application Ser. No. 16/352,741, filed on Mar. 13, 2019 which is incorporated herein by reference.

In some examples, the NORS can include a gelling agent to be a NORG, or the like. Non-limiting examples of suitable gelling agents can include xanthan gum starch, guar gum, locust bean gum, gum karaya, gum tragacanth, gum Arabic, cellulose derivatives, alginate, pectin, carrageenan, gelatin, gellan, agar, the like, or a combination thereof. The gelling agent can generally be present in the NORS in an amount to provide the NORS with a viscosity of from about 3,000 centipoise to about 150,000 centipoise. In some specific examples, the gelling agent can be added to the NORS in an amount of from about 0.5 wt % to about 2 wt %, from about 1 wt % to about 3 wt %, from about 2 wt % to about 4 wt %, or from about 3 wt % to about 5 wt % based on a total weight of the NORS.

As described above, in some examples, the NORS can be contained or housed in a container, which can be a pressurized container, or other suitable container. In some further examples, the NORS can be included with instructions regarding application of the NORS to a treatment area.

The present disclosure also describes therapeutic systems. The therapeutic systems can include a nitrite composition having a viscosity of from about 5,000 centipoise to about 100,000 centipoise and an acidifying component having a viscosity of from about 5,000 centipoise to about 100,000 centipoise.

The nitrite composition can include a nitrite agent, such as are described above. The nitrite agent can be present in the nitrite composition in a variety of amounts. For example, the nitrite agent can generally be present in the nitrite composition in an amount from about 0.02 wt % to about 20 wt % based on the total weight of the composition. In other examples, the nitrite agent can be present in the nitrite composition in an amount of from about 0.02 wt % to about 0.2 wt %, from about 0.2 wt % to about 2 wt %, from about 2 wt % to about 10 wt %, or from about 10 wt % to about 20 wt % based on a total weight of the nitrite composition. In some additional examples, the nitrite agent can be present in the nitrite composition in an amount of from about 0.3 millimolar (mM) to about 1000 mM. In other examples, the nitrite agent can be present in the nitrite composition in an amount of from about 0.2 mM to about 20 mM, from about 2 mM to about 20 mM, from about 10 mM to about 100 mM, from about 40 mM to about 400 mM, or from about 100 mM to about 1000 mM. In some examples, the nitrite composition can have a pH of from about 5 to about 8 prior to mixing with the acidifying composition.

The acidifying composition can include an acidifying agent, such as are described above. The acidifying agent can be included in the acidifying composition in a variety of amounts. For example, the acidifying agent can generally be included in the acidifying composition in an amount to achieve a pH of from about 2.5 to about 5, or from about 3 to about 4 when mixed with the nitrite composition to prepare a NORS. Prior to mixing the acidifying composition can generally have a pH of from about 2 to about 5. In some specific examples, the acidifying agent can be present in the acidifying composition in an amount of from about 0.02 wt % to about 40 wt % based on a total weight of the acidifying composition. In some specific examples, the acidifying agent can be included in the acidifying composition in an amount of from about 0.02 wt % to about 0.2 wt %, from about 0.2 wt % to about 2 wt %, from about 2 wt % to about 10 wt %, from about 10 wt % to about 20 wt %, or from about 20 wt % to about 40 wt % based on a total weight of the acidifying composition. In some additional examples, the acidifying agent can be present in the acidifying composition in an amount of from about 0.2 millimolar (mM) to about 1000 mM. In other examples, the acidifying agent can be present in the acidifying composition in an amount of from about 0.2 mM to about 20 mM, from about 2 mM to about 20 mM, from about 10 mM to about 100 mM, from about 40 mM to about 400 mM, or from about 100 mM to about 1000 mM.

The nitrite composition and the acidifying composition can independently include an aqueous carrier. The aqueous carrier can be independently selected from water, phosphate-buffered saline (PBS), Dulbecco's PBS, Alsever's solution, Tris-buffered saline (TBS), balanced salt solutions (BSS), such as Hank's BSS, Earle's BSS, Grey's BSS, Puck's BSS, Simm's BSS, Tyrode's BSS, BSS Plus, Ringer's lactate solution, normal saline (i.e. 0.9% saline), ½ normal saline, the like, or a combination thereof.

In some examples, one or more of the nitrite composition and the acidifying composition can include a gelling agent. Non-limiting examples of gelling agents can include xanthan gum starch, guar gum, locust bean gum, gum karaya, gum tragacanth, gum Arabic, cellulose derivatives, alginate, pectin, carrageenan, gelatin, gellan, agar, the like, or a combination thereof. The gelling agent can be added in various amounts, depending on the formulation. In some examples, the gelling agent can be added to the nitrite composition and/or the acidifying composition in an amount of from about 0.5 wt % to about 5 wt % based on a total weight of the individual composition. In other examples, the gelling agent can be added to the nitrite composition and/or the acidifying composition in an amount of from about 1 wt % to about 3 wt % based on a total weight of the individual composition. In still other examples, the gelling agent can be added to the nitrite composition and/or the acidifying composition in an amount of from about 0.5 wt % to about 2 wt %, from about 1 wt % to about 3 wt %, from about 2 wt % to about 4 wt %, or from about 3 wt % to about 5 wt % based on a total weight of the individual composition.

It is also noted that one or more of the nitrite agent and the acidifying agent can be encapsulated in the therapeutic system. In some examples, the nitrite agent can be encapsulated. In some examples, the acidifying agent can be encapsulated.

As described above, the nitrite composition and the acidifying composition can be combined together to form a NORS. However, in the example of the therapeutic system, the nitrite composition and the acidifying composition can be maintained separately to prevent premature NO generation prior to application. Accordingly, in some examples, the therapeutic system can include a container. In some examples, the container can be a dispensing container. In some cases, the nitrite composition and the acidifying composition can be separated from one another in separate containers or separate compartments of the same container. In some specific examples, the nitrite composition can be housed or contained in a first compartment of a container and the acidifying composition can be housed or contained in a second compartment of the container. In other examples, the nitrite composition can be housed or contained in a first container and the acidifying composition can be housed or contained in a second container. In some specific examples, the nitrite composition and the acidifying composition can be included in separate compartments of a common container, but where the individual compositions are mixed during or upon dispensing of the compositions. For example, in some cases, the nitrite composition and the acidifying composition can be included in separate compartments of the same container and can be dispensed through a cork-screw, or similar, dispenser to facilitate mixing during dispensing. In other examples, the nitrite composition and the acidifying composition can be separately dispensed from a common container or separate containers to a treatment area and manually mixed at the treatment area.

In some examples, the nitrite composition and the acidifying composition can be packaged together in a common package, whether or not they are included in a common container, separate containers, or a combination thereof. In some further examples, the package can further include instructions regarding application of the nitrite composition and the acidifying composition to the treatment area. The instructions can vary depending on the particular type of therapeutic system being employed. In some embodiments, the nitrite and acidifying compositions can be pre-mixed so that the solution/gel is activated, but kept in a container that eliminates or minimizes the presence of oxygen. Thus, a reaction between the nitrite and the acidifier can be limited or prevented. For example, a bag on valve device/system, or the like, can be used.

In some embodiments, the NORS compositions can include or can be otherwise combined or co-administered with a supplemental active agent. Supplemental or additional active agents can include any active or therapeutic agent that is suitable for administration for a given indication related or complimentary to the indication for which the NO is administered. Exemplary supplemental or additional active agents can include without limitation, antifungal agents, anthelmintic agents, antiviral agents, antibacterial agents, antiseptics, prebiotics, probiotics, vitamins, analgesics agents, anti-inflammatories, antioxidants, desquamating agents, among others. Additionally, other agents or ingredients that aid or otherwise assist the effectiveness of NO and/or any supplemental active agents can be included in the NORS or co-administered therewith, such as penetration enhancers, emollients, thickeners, lubricants, softeners, adjuvants, etc.

The present disclosure also describes medicament containment devices that can be used in connection with the NORS, the therapeutic systems, or other suitable medicaments. The medicament containment devices can include a housing having an interior wall forming or defining a containment reservoir configured to contain nitric oxide therein when the device is attached to a subject. The device can also include an attachment feature or member configured to attach the device to a subject.

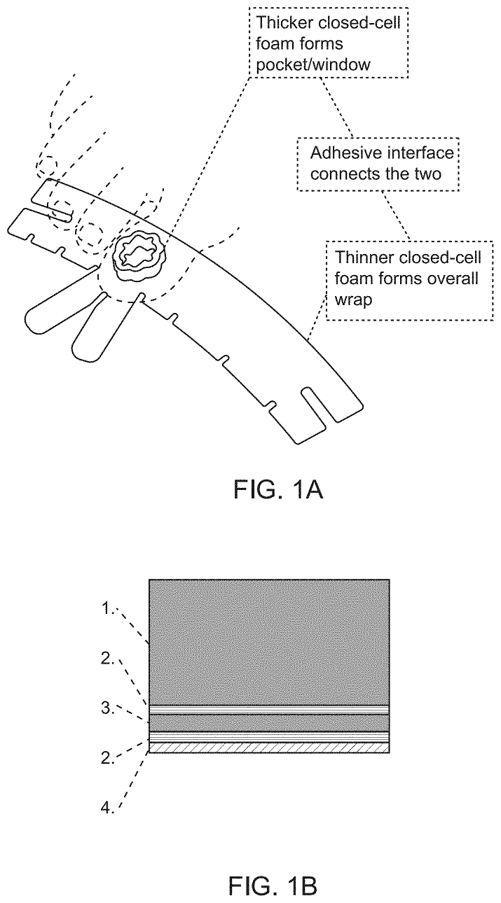

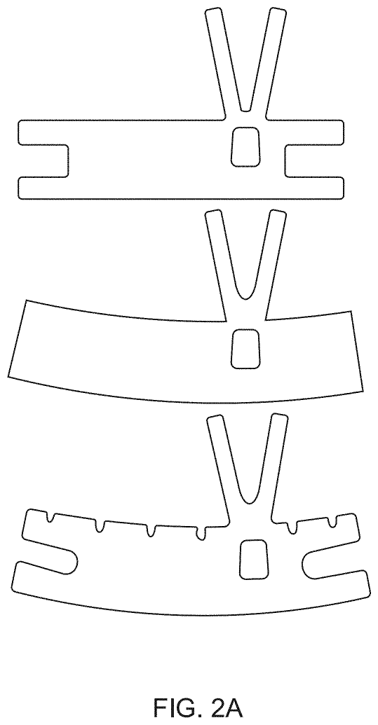

Turning to the figures, FIG. 1A illustrates one non-limiting example of a medicament containment device. The particular device illustrated in FIG. 1 is designed for placement about a phalange or digit of the foot. As can be seen in FIG. 1A, the medicament containment device includes a housing including a hollow channel or chamber for placement over a nail of the toe. The toenail and surrounding tissues can form a basement substrate of the containment reservoir when the device is positioned on the subject and the interior walls of the hollow channel or chamber can define the inner diameter or walls of the containment reservoir. The housing of the device presented in FIG. 1A also includes lateral straps for wrapping laterally about the circumference of the toe and tip straps (bunny ear straps) for wrapping about the tip of the toe. In some examples, a portion of the longer side of the lateral strap can be wrapped about the circumference of the toe and can further form a cap or closure to seal or close off the top of the containment reservoir opposite the toenail. Thus, the medicament containment device can provide a closed device or system by forming an enclosed containment reservoir to maintain effective concentrations of NO therein and focus NO delivery and therapy to a localized anatomical region while minimizing broader or systemic exposure to potentially toxic levels of NO. Localizing the medicament in this manner not only shields surrounding and distal physiology from NO exposure, but also concentrates NO exposure to an afflicted area of a subject's physiology (e.g. a toenail and surrounding tissue).

As illustrated in FIG. 1B, the medicament containment device can include various layers. Layer 1 can represent the containment reservoir. The layers 2 can represent adhesive layers for coupling various components of the housing together. Layer 3 can represent the strap portion of the housing to which the containment reservoir portion 1 is coupled via upper adhesive layer 2. Layer 4 can represent a release liner that is removable to expose lower adhesive layer 2 to facilitate coupling of the device to a subject.

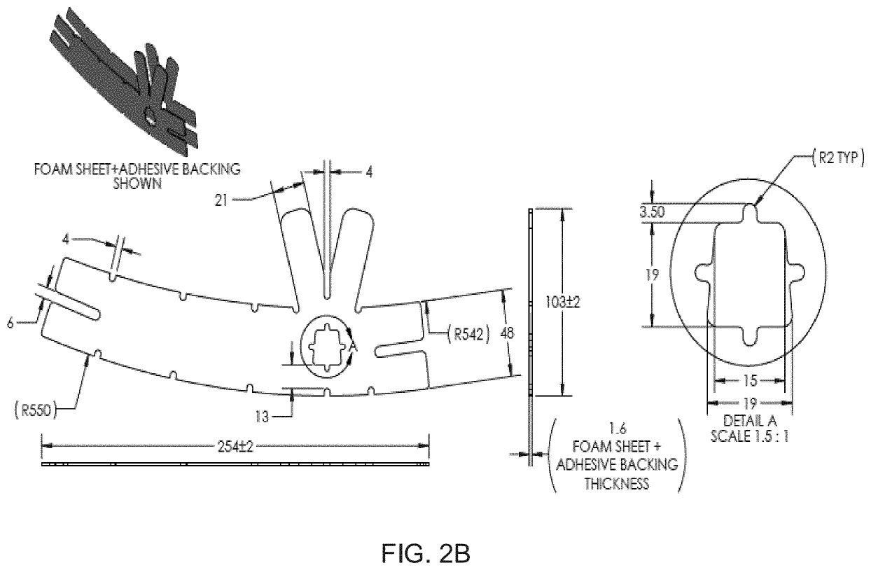

FIG. 2A illustrates various examples of a strap component of a medicament containment device. It is noted that a strap component is not required for all medicament containment devices, but it can be useful in some examples. It is further noted that the bunny ear straps or flaps are also not required in all designs. However, in some examples, the bunny ear straps or flaps can provide better securement and/or conformance about a phalange (e.g., a finger, a toe) during placement of the device, and can assist in retaining the device in place during a subject's physical activities, such as walking or running, handling things and bathing. In some examples, as illustrated in the top embodiment of the strap component of the device of FIG. 2A, cut-outs can be formed on the ends of the lateral straps to provide better securement and/or conformance of the device about a body part. In some additional examples, as illustrated in the middle embodiment of the strap component of the device of FIG. 2A, the strap component can have a slightly curved projection to provide a more conformal shape to the strap component to some body parts. In some further examples, as illustrated in the bottom embodiment of the strap component of the device of FIG. 2A, the strap component can include one or more relief cuts along one or more side edges of the strap component to provide further conformance of the strap component to some body parts.

FIG. 2B illustrates one specific example of a strap component of a medicament containment device. The measurements of the various components are provided in millimeters (mm). However, it is noted that the dimensions of the strap component are not particularly limited and can depend on the particular body part to which the device is being coupled, the size of the treatment area, etc.

Where included, the strap component can include or be made from a variety of materials. Non-limiting examples can include polyethylene, polyurethane, silicone, neoprene, ethylene propylene diene monomer rubber (EPDM), styrene-butadiene rubber (SBR), the like, or a combination thereof. In some examples, the strap can be coated with a protective material that is chemically compatible with NO or is otherwise inert so that it won't react with NO. In some further examples, the strap component can be made of a foam material.

In some additional examples, the strap can include an adhesive for application of the strap component to a body part of a subject. Non-limiting examples of suitable adhesives can include acrylic adhesives, cyanoacrylic adhesives, silicone adhesives, polyurethane adhesives, epoxies, the like, or a combination thereof.

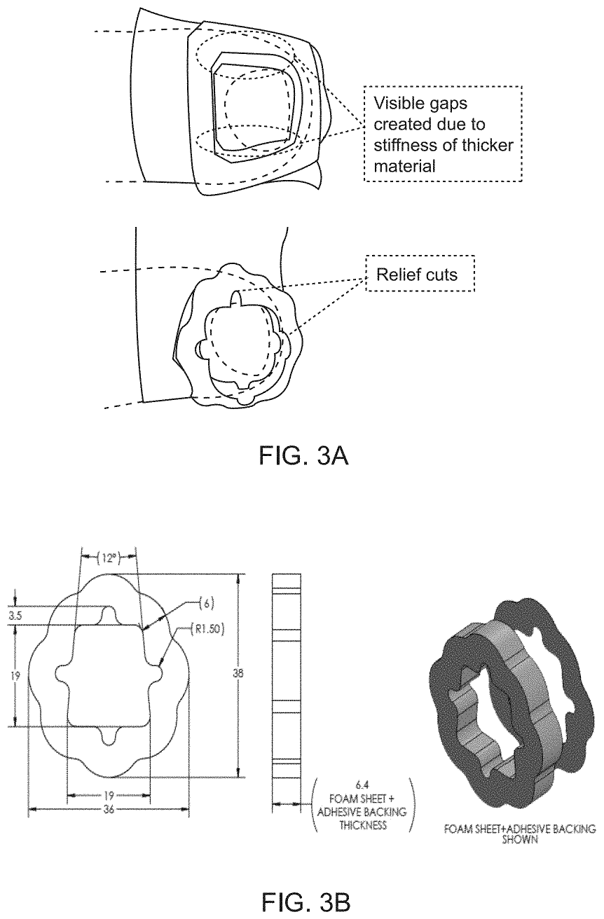

FIG. 3A illustrates non-limiting examples of a containment reservoir for a medicament containment device. As illustrated in the examples of FIG. 3A, the containment reservoir can have a variety of shapes and configurations. For example, as illustrated in the bottom example of FIG. 3A, the containment reservoir can be shaped to conform to or otherwise target the treatment area. This can be accomplished with relief cuts, a tailored shape of the containment reservoir, the like, or a combination thereof.

FIG. 3B illustrates one specific example of a containment reservoir of a medicament containment device. The measurements of the various components and features are provided in millimeters (mm). However, it is noted that the dimensions of the medicament containment device are not particularly limited and can depend on the particular body part to which the device is being coupled, the size of the treatment area, etc. In some examples, the containment reservoir can have a volume of from about 1 ml to about 20 ml (e.g. when positioned on a body part of a subject). In one embodiment, a device/system as recited herein may have a reservoir with a skin or tissue contact surface area that is up to about 914 cm² in size. In another embodiment, a device/system as recited herein may have a reservoir with a depth of up to about 1 cm. In other embodiments, the depth may be up to about 0.16 cm. In yet other embodiments, a device/system as recited herein can have a reservoir with a volume of up to about 300 ml. In another embodiment, the volume can be up to about 146 ml.

As illustrated in FIG. 3B, the containment reservoir can be formed of an individual component that is attachable directedly to a subject or to a strap component or other base component. While not specifically illustrated, in some examples, the containment reservoir can form part of the strap component (e.g. be formed integrally therewith, rather than provided as a separate attachable component), or the like, such that an inner wall of the strap component, or the like, defines or forms a wall of the containment reservoir without requiring any additional components to be attached to form the containment reservoir. In some further examples, the containment reservoir can include an adhesive. Non-limiting examples of adhesives can include acrylic adhesives, cyanoacrylic adhesives, silicone adhesives, polyurethane adhesives, epoxies, the like, or a combination thereof.

In some examples, the containment reservoir can include a hollow channel or cavity that is suitable for receiving a NORS, or a nitrite composition and an acidifying composition, or other suitable medicament thereto after attaching the device to a body part of a subject. In other examples, the containment reservoir can be preloaded with a NORS, a nitrite component and an acidifying component, or other suitable medicament prior to application of the device to the subject.

In one specific example, the basement or substrate of the containment reservoir can be initially formed with a water-soluble membrane (e.g., polyvinyl alcohol membrane, for example). An inactivated NORS including a nitrite agent and an acidifying agent in a dry form can be supplied in a lower compartment between the water-soluble membrane and a frangible membrane. An aqueous carrier can be supplied in an upper compartment between the frangible membrane and a cap of the containment reservoir positioned opposite the water-soluble membrane. The water-soluble membrane is configured to be positioned against the treatment area. Thus, when the device is positioned over the treatment area, pressure can be applied to the cap to cause the frangible membrane to rupture. The aqueous carrier can then flood the lower compartment including the nitrite agent and the acidifying agent to form an activated NORS and begin generation of NO. Further, the aqueous carrier can dissolve the water-soluble membrane to directly expose the treatment area to the NORS. Other suitable methods of pre-loading the NORS, the nitrite agent and the acidifying agent, or other suitable medicament to the containment reservoir can also be employed.

The containment reservoir can further include a cap or closure. In some examples, where the medicament containment device includes a preloaded NORS, the like, or other suitable medicament, the cap or closure can be pre-attached to the medicament containment device. In other examples, where the containment reservoir is a hollow channel or cavity, a portion of the medicament containment device, such as a strap or base portion, can be folded over the containment reservoir to form a cap or closure. In yet other examples, a separate cap portion can be supplied to apply over the containment reservoir after introduction of the medicament therein.

The medicament containment device can be adapted to attach to a variety of body parts of a subject. Non-limiting examples can include a phalange (e.g. finger or toe), an arm, a hand, a leg, a foot, a torso, a head, a penis, a nipple, a neck, the like, or a combination thereof.

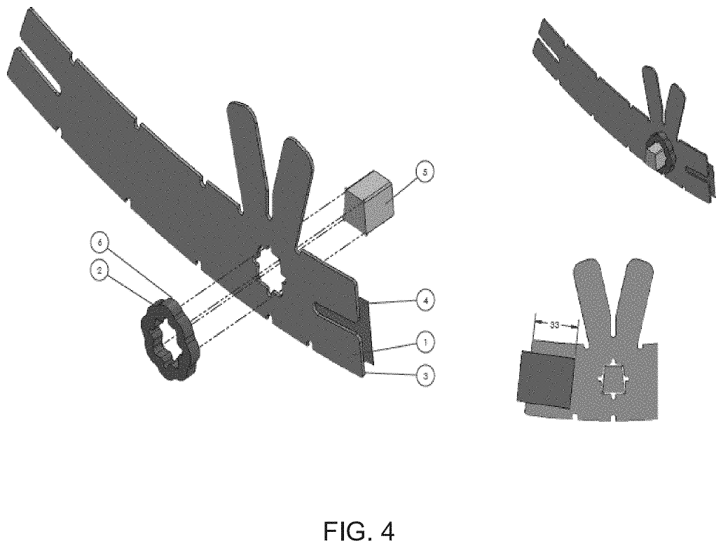

FIG. 4 illustrates one non-limiting example of a method of manufacturing a medicament containment device. As illustrated in FIG. 4, a base portion or strap portion 1 can include a release line 4 removably adhered thereto. The base portion or strap portion 1 can include a cut region corresponding to the containment reservoir. An alignment block 5 can be inserted into the cut region to guide the containment reservoir 2 into proper alignment on the base portion or strap portion 1. The containment reservoir 2 can include an adhesive 6 to facilitate attachment of the containment reservoir 2 to the strap portion 1. After attachment of the containment reservoir 2 to the strap portion 1, the alignment block 5 can be removed. Additionally, the strap portion 1 can include an adhesive 3 to facilitate attachment of the assembled device to a subject.

The present disclosure also describes methods of treating or alleviating a variety of topical conditions, such as infections (e.g. fungal, bacterial, or viral infections), inflammation, wounds, scrapes, punctures, lacerations, burns (including sunburns), psoriasis, acne, dermatitis, pilaris keratosis, warts, etc. that are responsive to nitric oxide (NO) treatment in a subject. The method of treating a topical condition can include applying a medicament containment device to a subject. The device can form a containment reservoir about a treatment area. The method can further include introducing NO (e.g. with a NORS) into the containment reservoir. The medicament containment device can be configured to substantially contain the NORS/NO within the containment reservoir.

The methods can be used to treat a variety of topical infections. In some examples, the topical infection can be a skin infection. Non-limiting examples of skin infections can include a wart, molluscum contagiosum, ringworm, a carbuncle, a boil, impetigo, a pilondil cyst, sporotrichosis, shingles, the like, or a combination thereof. In some examples, the topical infection can be a nail infection. Non-limiting examples of nail infections can include onychomycosis, green nail syndrome, the like, or a combination thereof. In some specific examples, the methods can be used to treat onychomycosis.

The medicament containment device can be applied to the subject in a variety of ways. In some examples, the medicament containment device can be applied via an adhesive. In other examples, the medicament containment device can be applied by wrapping the device about a portion of a body part of a subject, such as a hand, a foot, an arm, a leg, a torso, a phalange, a penis, a head, a neck, or a combination thereof. In other examples, the medicament containment device can be applied to a subject by a combination of methods, such as via wrapping, an adhesive, a hook and loop fastener, a magnetic attachment, a buckle, a clip, a cinch, the like, or a combination thereof.

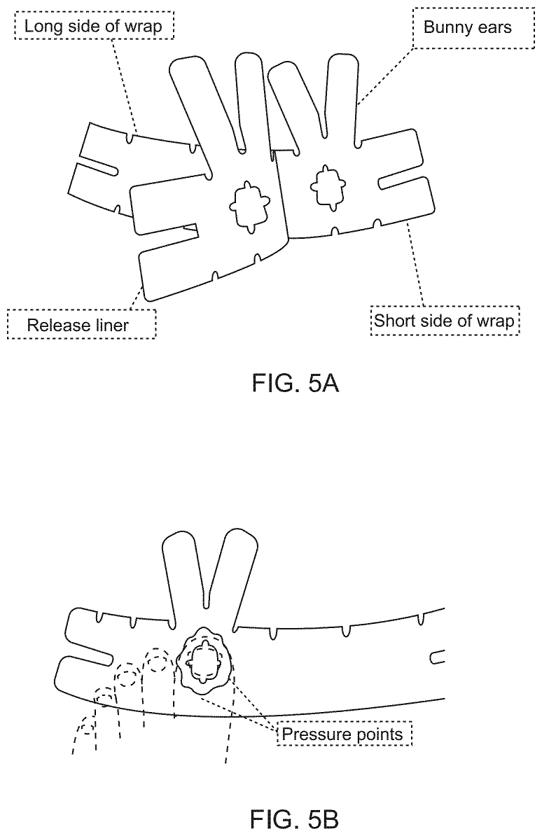

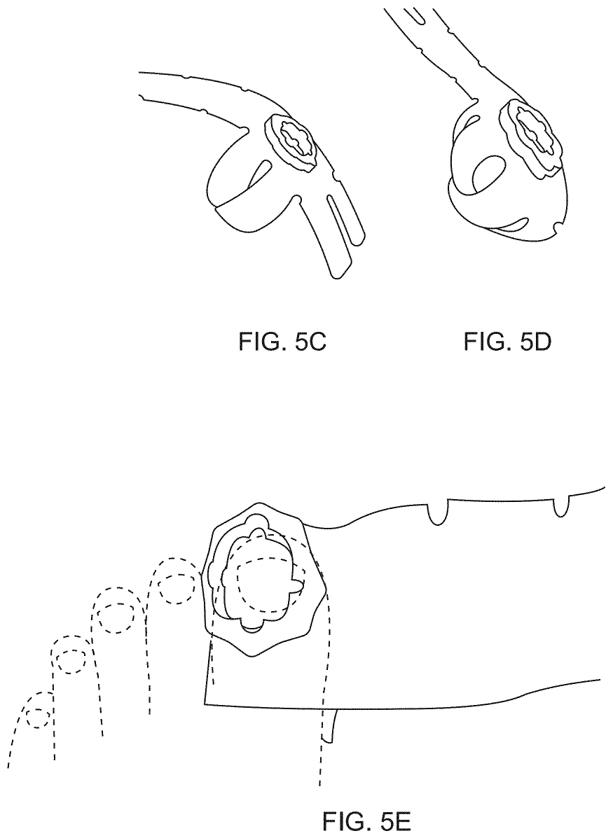

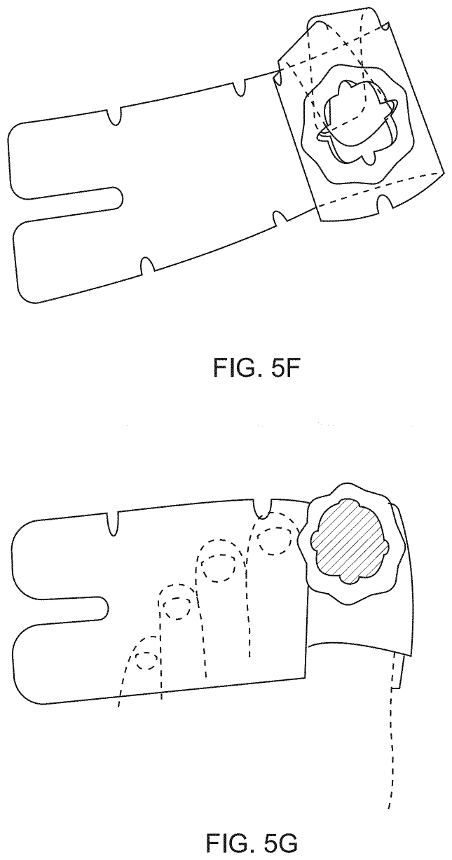

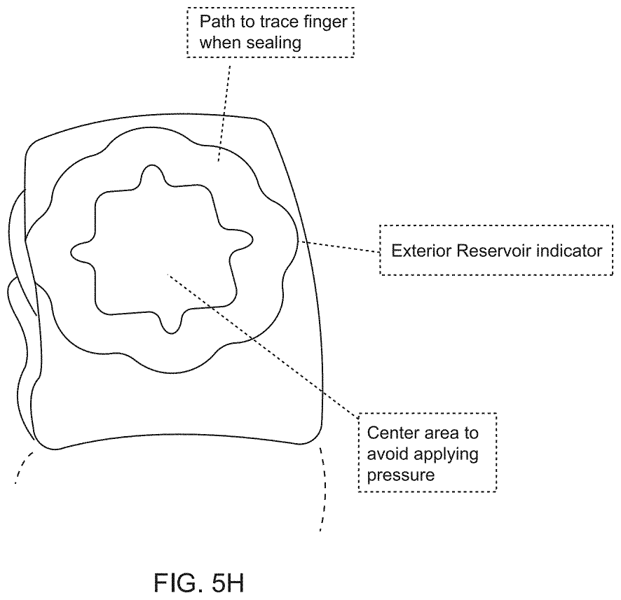

FIGS. 5A-5I illustrate various possible steps in a method of applying a medicament containment device to a subject. For example, FIG. 5A illustrates removing a release liner from the medicament containment device prior to application to a subject to expose an adhesive on the application side of the device. FIG. 5B illustrates initial application of the medicament containment device to a portion of a body part (e.g., a toe) of a subject. In this example, the toe nail and surrounding tissues form the target treatment area. Accordingly, pressure is applied to adhere the device to the toe so that the containment reservoir circumscribes the treatment area and to form an adhesive seal to contain NO within the containment reservoir.

FIG. 5C illustrates how the optional bunny ears can be folded down over the tip of the phalange to render the device more conformal to the toe. FIG. 5D illustrates how a short end of the strap portion of the device can then be folded over the bunny ears. These portions can also include adhesive to adhere to the phalange. FIG. 5E illustrates the appearance of the device on the toe after applying the bunny ears and the short side of the strap component.

FIG. 5F illustrates how the long side of the strap component can be folded under the toe and be folded to cover the bunny ears and short side of the strap component underneath the toe. FIG. 5G illustrates how this appears when applied to the toe. Further, FIG. 5G also illustrates a NORS having been applied within the containment reservoir to directly contact the treatment area, with the nail and surrounding tissues forming the basement of the containment reservoir. FIG. 5H then shows how the long portion of the strap can be folded over the top of the containment reservoir to cap the containment reservoir and form yet another adhesive seal to contain the NO within the containment reservoir. Additionally, FIG. 5H shows an exterior reservoir indicator which can be a symbol or emblem or other marking which substantially aligns with the perimeter of the container reservoir which is no covered underneath the long end of the wrap in the closed state. The exterior reservoir indicator can allow a subject to interact with the device in a way that does not disturb and/or protects the contents of the containment reservoir. For example, the exterior reservoir indicator can have an outer portion indicating where a subject should apply pressure in order to seal the perimeter of the containment reservoir against the overlapping long end of the wrap. Furthermore, the exterior reservoir indicator can allow identification of the chamber of the containment reservoir so that the subject can avoid applying pressure to the device in a manner that might dislodge or negatively impact the NORS that has been dispensed into the containment reservoir. The exterior reservoir indicator can take any shape, size, or include any markings, signs, words, or directions, needed to accommodate a particular size or shape of containment reservoir and/or to alert the subject as to proper use of the device.

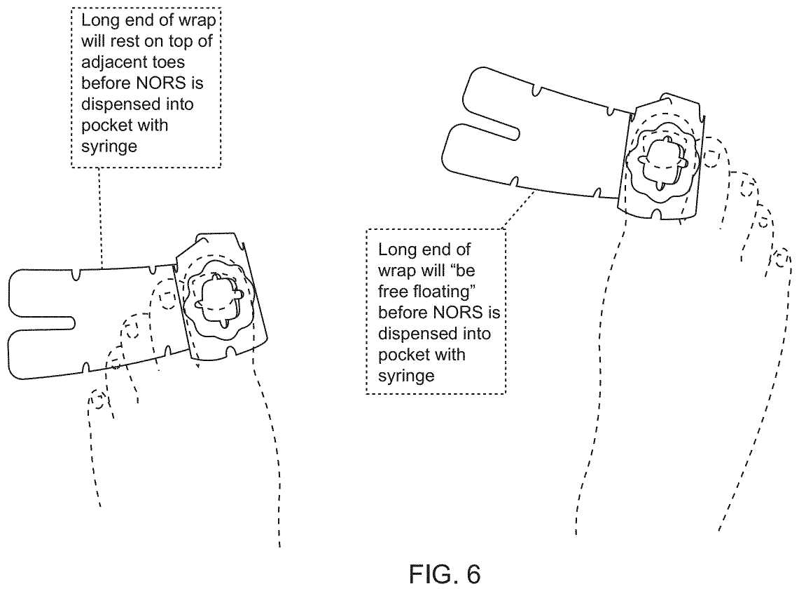

FIG. 6 presents comparative examples of how the device presented in FIGS. 5A-5H would appear on the left foot vs. the right foot. In either case, the long side of the strap can be folded over the containment reservoir to cap the containment reservoir and form an adhesive seal to contain the NO within the containment reservoir.

NO can be introduced into the containment reservoir in a variety of ways. In some examples, a nitrite composition and an acidifying composition can be mixed prior being introduced into the containment reservoir. In some specific examples, the nitrite composition and the acidifying composition can be mixed as they are dispensed into the containment reservoir. In other examples, the nitrite composition and the acidifying composition can be mixed and subsequently dispensed into the containment reservoir, or they can be mixed within the treatment reservoir. In some specific examples, a NORS can be pre-loaded into a container that can be pressurized to minimize NO production prior to dispensing and subsequently dispensed into the containment reservoir. In other examples, the containment reservoir can include an inactive NORS preloaded therein and that can be subsequently activated after placement at the treatment area to generate NO.

A therapeutically effective amount of NO can be introduced into the containment unit. In one example, a therapeutically effective amount of NO can be from about 1 ppm to about 10,000 ppm NO. In another example, a therapeutically effective amount can be from about 40 ppm to about 1000 ppm NO. In another example, the therapeutically effective amount of NO can be from about 4 ppm to about 400 ppm NO. In another example, the therapeutically effective amount of NO can be from about 100 ppm to about 220 ppm NO. In another example, the therapeutically effective amount is from about 50 ppm to about 200 ppm NO. In one specific example, the therapeutically effective amount can be about 160 ppm NO. In another example, the therapeutically effective amount can be less than or equal to 160 ppm NO.

Where the NO is introduced via a NORS, the NORS can generally be applied to the treatment area in an amount of from about 0.5 milliliter (ml) to about 40 ml. In other examples, the NORS can be applied to the treatment area in an amount from about 1 ml to about 30 ml, from about 5 ml to about 20 ml, or from about 10 ml to about 20 ml. In other examples, the NORS can be applied to the treatment area in an amount from about 0.5 ml to about 5 ml, from about 1 ml to about 10, from about 5 ml to about 15 ml, from about 10 ml to about 20 ml, from about 15 ml to about 25 ml, from about 20 ml to about 30 ml, from about 25 ml to about 35 ml, or from about 30 ml to about 40 ml. In still other examples, the NORS can be applied to the treatment area in an amount of from about 0.5 gram (g) to about 40 g. In other examples, the NORS can be applied to the treatment area in an amount from about 1 g to about 10 g, from about 5 g to about 15 g, from about 10 g to about 20 g, from about 15 g to about 25 g, from about 20 g to about 30 g, from about 25 g to about 35 g, or from about 30 g to about 40 g.

The present disclosure also describes a treatment system. The treatment system can include a medicament containment device as described herein and a dispensing device. The dispensing device can be a pump dispenser, a single-barreled syringe, a double-barreled syringe, or the like. Where a double-barreled syringe is used, the double-barreled syringe can include a tip that is configured to mix individual components, such as a nitrite component and an acidifying component, as they are dispensed.

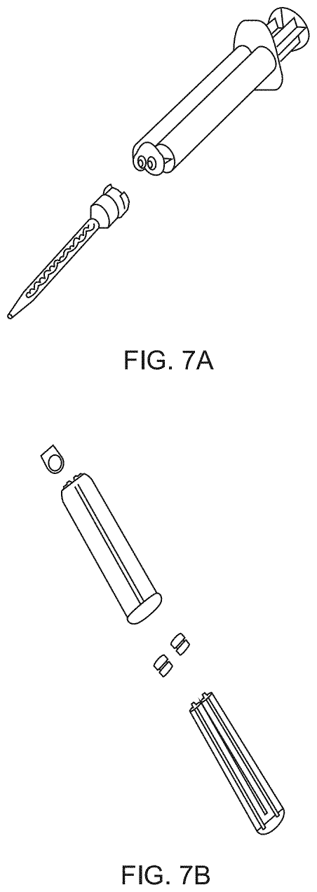

One non-limiting example of a dispensing device is presented in FIG. 7A. Specifically, the dispensing device includes a double-barreled syringe 1 and a corkscrew mixing tip 2 configured to mix individual components, such as a nitrite composition and an acidifying composition, as they are dispensed. FIG. 7B presents additional details regarding the double-barreled syringe. Specifically, the double-barreled syringe can include a housing, a plunger that slidably engages the housing from point A to point B, and vice versa, and plunger seals that are chemically compatible with the individual components included in the separate chambers of the syringe. A syringe cap can also be included to seal or otherwise enclose the dispensing end of the syringe.

In some examples, the treatment system can also include a therapeutic system as described herein. In yet other examples, the treatment system can also include a NORS as described herein.

### EXAMPLES

### Example 1—Treatment of Onychomycosis with Nitric Oxide Releasing Gel

Onychomycosis (OMC) is a common fungal infection of the toe nail caused by dermatophyte fungi causing white or yellow nail discoloration, thickening of the nail, and separation of the nail from the nail bed. OMC is a common nail disease, affecting over 35 million people in the United States. It represents about half of all nail diseases. The global prevalence of onychomycosis is 10% of all adults. This percentage increases to 20% of adults who are age 60 or older. Systemic antifungals are the most effective treatment, with meta-analyses showing mycotic cure rates of around 70% for terbinafine, 60% for itraconazole and 50% for fluconazole. Oral administration causes systemic side effects such as nausea, dizziness, vomiting or liver damage. Many patients are not candidates due to the potential liver damage. Trichophyton resistance to terbinafine treatment is an emerging problem. There is an avid demand for better topicals to treat this disease.

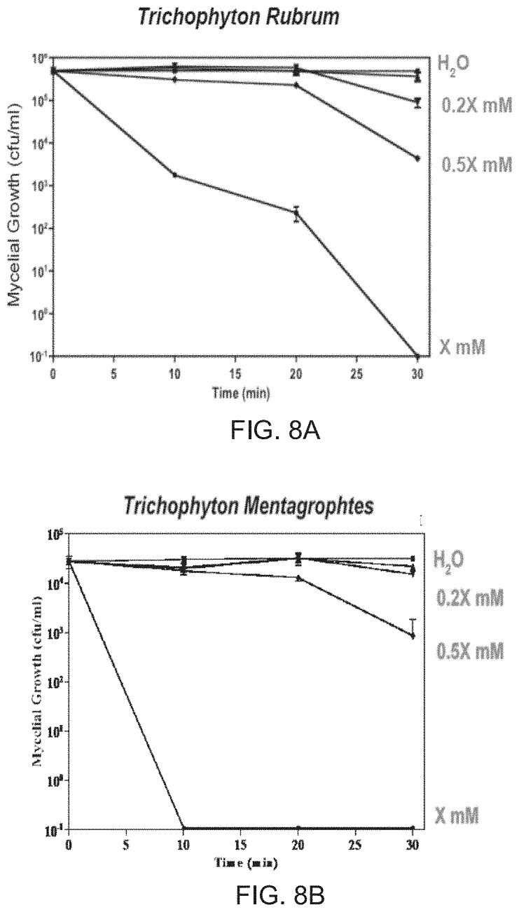

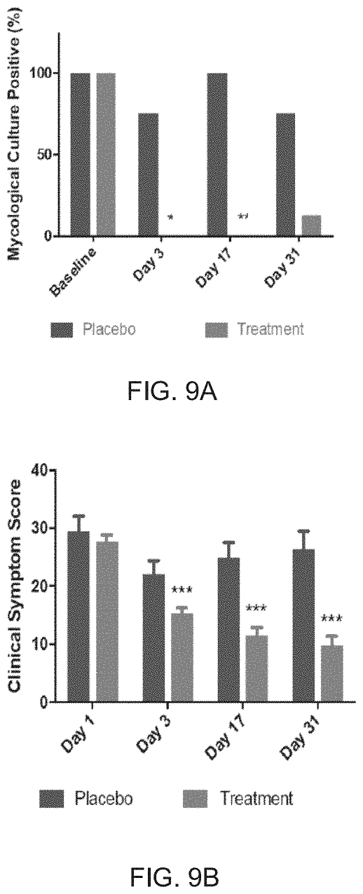

In vitro studies were performed at 10-30 min exposures to nitric oxide releasing solutions/gels. As illustrated in FIGS. 8A and 8B, NO can completely eradicate the fungi associated with athlete's foot, causing a complete kill of mycelia and conidia. Further, in human clinical trials, NORS demonstrated significant fungicidal activity as well as anti-inflammatory effect on subjects with athlete's foot (See FIGS. 9A and 9B).

Additionally, a blinded, ex vivo study, of 15 volunteers with suspected distal subungual onychomycosis in the great toenail (OMCGN) were evaluated. NORG was evaluated as a fungicidal as compared to an incumbent comparator (10% Efinaconazole). Toe nail clippings were obtained during a clinical visit to a podiatrist clinic. Half the clippings were sent to an independent commercial laboratory for determination of a positive OMC diagnosis as determined by presence of fungal bodies (KOH), viable and fungal (culture). The remaining half of the sample was sent to a University Laboratory for testing. The sample was divided. Half was cultured for fungal growth. The remaining sample was exposed to either 10% Efinaconazole (continuously) or NORG-80 (8 hours daily) for 7 consecutive days. Following exposure, samples were shredded to expose the interior of the nail clippings and plated to determine fungal growth. Evaluable samples required a positive culture from the independent lab of KOH and mycological culture AND a positive mycological culture of the baseline sample prior to treatment intervention. Mycotic cure was defined as no fungal growth after 21 days post treatment intervention.

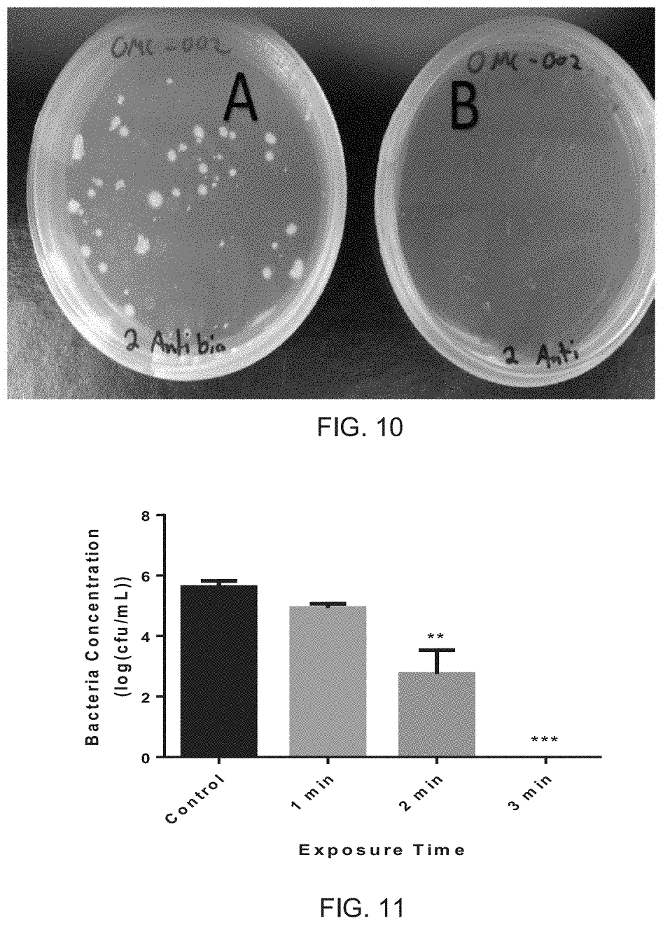

15 subjects were enrolled, two samples met protocol criteria for analysis and both were in the NORG-80 cohort. NORG-80 was fungicidal. The nail clippings became orange red (See FIG. 10) presumably due to a known oxidation process resulting in the color change. This color change helped to demonstrate that the nitric oxide penetrated through the entire thickness of the nail tissue.

Based on these findings, it appears that NORG-80 is able to penetrate the toe nail matrix of a big toe (e.g. big toe nail) and eliminate the fungi associated with OMC.

### Example 2—Exposure of C. acnes to NORS

Approximately 10⁵ cfu/ml of C. acnes was exposed to 60 mM NORG. No bacteria detection was found after 3 minutes. n=3 (**=p<0.01, ***=p<0.001). (See FIG. 11).

### Example 3—Comparison of NORS and NORG

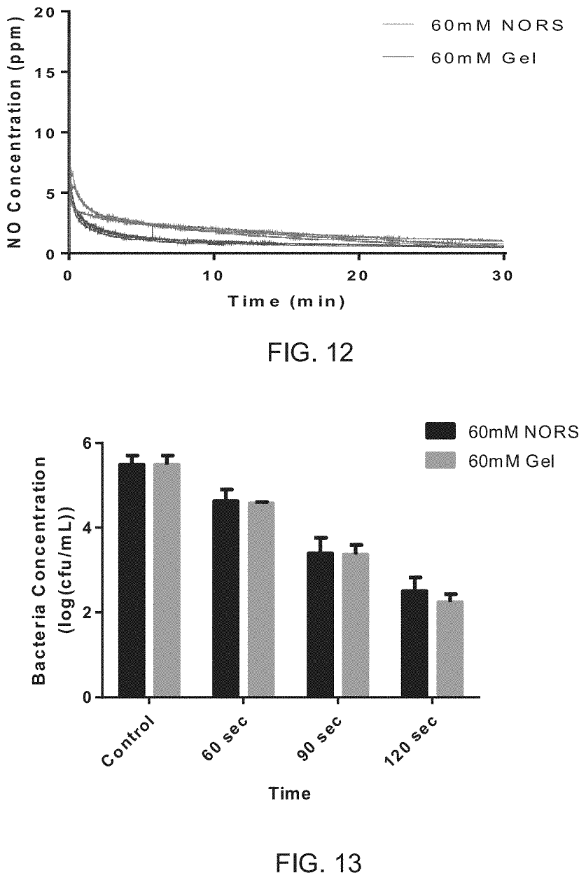

A 60 mM nitric oxide releasing solution (NORS) and a 60 mM nitric oxide releasing gel (NORG) were prepared to compare nitric oxide release from each. Approximately 1 ml of each composition at pH 3.5 was introduced into a blow-over device connected to a chemiluminescence detector to measure NO production. Nitrogen was used as the carrier gas at a flow rate of 1 L/min. (See FIG. 12 and Table 1).

TABLE 1
Results of NORS vs NORG Comparison

| | Nitric Oxide | Nitric Oxide |
|---|---|---|
| | Releasing Solution | Releasing Gel |
| Area Under the Curve | 51.52 | 29.32 |
| (ppm*min) | | |
| Slope after 1 min. | −0.089 | −0.054 |
| (ppm/min) | | |
| NO Measurement | 779 | 516 |
| after 30 min. (ppb) | | |

Additionally, FIG. 13 illustrates the concentration of S. aureus after exposure to 60 mM NORS and 60 mM NORG at pH 3.5. More specifically, approximately 10⁵ cfu/mL of bacteria was placed into the sample and was vortexed for 5 seconds. After the exposure time ended the reaction was neutralized with NaOH. The sample was then vortexed for 5 seconds, diluted, and plated. No significance was found at each time point between the solution and gel.

### Example Embodiments

The following examples pertain to specific technology embodiments and point out specific features, elements, or steps that may be used or otherwise combined in achieving such embodiments.

In one example there is provided a method of treating a topical condition responsive to nitric oxide (NO) treatment in a subject, comprising applying a medicament containment device to a subject, said device forming a containment reservoir about a treatment area; and introducing a nitric oxide releasing substance (NORS) into the containment reservoir, wherein the medicament containment device is configured to substantially contain NO produced by the NORS within the containment reservoir.

In one example of a method of treating a topical condition responsive to NO therapy, wherein the topical condition is a skin infection.

In one example of a method of treating a topical condition responsive to NO therapy, the skin infection comprises a wart, molluscum contagiosum, ringworm, a carbuncle, a boil, impetigo, a pilondil cyst, sporotrichosis, shingles, eczema, or a combination thereof.

In one example of a method of treating a topical condition responsive to NO therapy, the topical infection is a nail infection.

In one example of a method of treating a topical condition responsive to NO therapy, the nail infection comprises onychomycosis, green nail syndrome, or a combination thereof.

In one example of a method of treating a topical condition responsive to NO therapy, the medicament containment device is applied via an adhesive.

In one example of a method of treating a topical condition responsive to NO therapy, the medicament containment device is applied by wrapping the device about a portion of a body part of a subject.

In one example of a method of treating a topical condition responsive to NO therapy, the body part is a hand, a foot, an arm, a leg, a torso, a phalange, a penis, a nipple, a head, a neck, or a combination thereof.

In one example of a method of treating a topical condition responsive to NO therapy, the containment reservoir has a volume of from about 0.1 ml to about 20 ml.

In one example of a method of treating a topical condition responsive to NO therapy, the NORS is introduced in an inactive state.

In one example of a method of treating a topical condition responsive to NO therapy, the NORS is prepared and activated as it is introduced into the containment reservoir.

In one example of a method of treating a topical condition responsive to NO therapy, the NORS is introduced as a gel.

In one example of a method of treating a topical condition responsive to NO therapy, the NORS is activated within the containment reservoir.

In one example of a method of treating a topical condition responsive to NO therapy, the NORS is formulated to release from about 10 ppm NO to about 5000 ppm NO.

In one example of a method of treating a topical condition responsive to NO therapy, the NORS is formulated to release NO for a period of from about 30 minutes to about 12 hours.

In one example, there is provided a nitric oxide releasing substance (NORS), comprising a nitrite agent; an acidifying agent; and an aqueous vehicle, wherein the NORS is formulated to release from about 1 ppm NO to about 10,000 ppm NO over a period of from about 1 second to about 48 hours.

In one example of a NORS, the nitrite agent comprises sodium nitrite, potassium nitrite, barium nitrite, calcium nitrite, nitrite orotate, amyl nitrite, magnesium nitrite, or a combination thereof.

In one example of a NORS, the nitrite agent is present in the NORS in an amount from about 0.01 wt % to about 10 wt %.

In one example of a NORS, the acidifying agent comprises ascorbic acid, ascorbyl palmitate, salicylic acid, malic acid, lactic acid, citric acid, formic acid, benzoic acid, tartaric acid, hydrochloric acid, sulfuric acid, phosphoric acid, or a combination thereof.

In one example of a NORS, the acidifying agent is present in the NORS in an amount to achieve a pH of from about 2.5 to about 5.

In one example of a NORS, the nitrite agent and the acidifying agent are present in the NORS at a weight ratio of from about 1:10 to about 10:1.

In one example of a NORS, at least one of the nitrite agent and the acidifying agent is encapsulated by an encapsulant material.

In one example of a NORS, the encapsulant material is present at a weight ratio of from about 0.05:1 to about 10:1 with the nitrite agent, the acidifying agent, or a combination thereof.

In one example of a NORS, the nitrite agent is encapsulated.

In one example of a NORS, the acidifying agent is encapsulated.

In one example of a NORS, the NORS has a viscosity of from about 3000 centipoise (cps) to about 150,000 cps.

In one example there is provided a medicament containment and/or administration device, comprising a housing having an interior wall forming a containment reservoir configured to contain a nitric oxide releasing substance (NORS) therein when the device is attached to a subject; and an attachment member configured to attach the device to the subject.

In one example there is provided a medicament containment and/or administration device, the containment reservoir has a volume of from about 1 ml to about 20 ml.

In one example there is provided a medicament containment and/or administration device, the containment reservoir is a hollow channel extending through the housing.

In one example there is provided a medicament containment and/or administration device, the containment reservoir includes an inactivated nitric oxide releasing solution (NORS).

In one example there is provided a medicament containment and/or administration device, the attachment member comprises a strap configured to wrap about a portion of a body part of the subject.

In one example there is provided a medicament containment and/or administration device, the body part is a phalange.

In one example there is provided a medicament containment and/or administration device, the strap includes a laterally extending portion configured to wrap around a circumference of the phalange and a tip portion configured to wrap about a tip of the phalange.

In one example there is provided a medicament containment and/or administration device, a portion of the strap is configured to wrap about the containment reservoir to cap the containment reservoir.

In one example there is provided a medicament containment and/or administration device, the attachment member comprises an adhesive layer configured to couple the containment device to a target treatment situs of the subject.

In one example there is provided a medicament containment and/or administration device, the device further comprises a cap configured for placement over the containment reservoir to direct release of NO from the NORS toward the subject when the device is attached to the subject.

In one example, there is provided a therapeutic system, comprising a nitrite composition having a viscosity of from about 5000 cps to about 100,000 cps; and an acidifying composition having a viscosity of from about 5000 cps to about 100,000 cps.

In one example of a therapeutic system, the nitrite composition comprises a nitrite agent.

In one example of a therapeutic system, the nitrite agent comprises sodium nitrite, potassium nitrite, barium nitrite, calcium nitrite, nitrite orotate, amyl nitrite, magnesium nitrite, or a combination thereof.

In one example of a therapeutic system, the acidifying composition comprises an acidifying agent.

In one example of a therapeutic system, the acidifying agent comprises ascorbic acid, ascorbyl palmitate, salicylic acid, malic acid, lactic acid, citric acid, formic acid, benzoic acid, tartaric acid, hydrochloric acid, sulfuric acid, phosphoric acid, or a combination thereof.

In one example of a therapeutic system, the nitrite composition, the acidifying composition, or both comprise a gelling agent.

In one example of a therapeutic system, the gelling agent comprises xanthan gum starch, guar gum, locust bean gum, gum karaya, gum tragacanth, gum Arabic, cellulose derivatives, alginate, pectin, carrageenan, gelatin, gellan, agar, or a combination thereof.

In one example of a therapeutic system, the nitrite composition has a pH of from about 5 to about 8.

In one example of a therapeutic system, the acidifying composition has a pH of from about 2 to about 5.

In one example, there is provided a treatment system, comprising a medicament containment device according to a device as recited herein; and a dispensing device.

In one example of a treatment system, the dispending device is a syringe.

In one example of a treatment system, the syringe is a double-barreled syringe configured to mixed individual components during dispensing.

In one example of a treatment system, the system further comprises a therapeutic system as recited herein.

In one example of a treatment system, the nitrite composition and the acidifying composition of the therapeutic system are pre-loaded into separate compartments of the dispensing device.

In one example of a treatment system, the nitrite composition and the acidifying composition of the therapeutic system are pre-loaded into separate dispensing devices.

In one example of a treatment system, the system further comprises a NORS as recited herein.

In one example of a treatment system, the NORS is pre-loaded into the dispensing device.

In one example of a treatment system, the dispensing device is pressurized to minimize NO production of the NORS prior to dispensing.

While the forgoing examples are illustrative of the principles of the present technology in one or more particular applications, it will be apparent to those of ordinary skill in the art that numerous modifications in form, usage and details of implementation can be made without the exercise of inventive faculty, and without departing from the principles and concepts of the technology.

What is claimed is:

1. A method of treating a topical condition responsive to nitric oxide (NO) therapy in a subject, comprising:
applying a medicament containment device to a subject, said device forming a containment reservoir about a treatment area; and
introducing a nitric oxide releasing substance (NORS) into the containment reservoir, wherein the medicament containment device is configured to substantially contain NO produced by the NORS within the containment reservoir.

2. The method of claim 1, wherein the topical condition is a skin infection.

3. The method of claim 2, wherein the skin infection comprises a wart, molluscum contagiosum, ringworm, a carbuncle, a boil, impetigo, a pilondil cyst, sporotrichosis, shingles, eczema, or a combination thereof.

4. The method of claim 1, wherein the topical infection is a nail infection.

5. The method of claim 4, wherein the nail infection comprises onychomycosis, green nail syndrome, or a combination thereof.

6. The method of claim 1, wherein the medicament containment device is applied via an adhesive.

7. The method of claim 1, wherein the medicament containment device is applied by wrapping the device about a portion of a body part of a subject.

8. The method of claim 7, wherein the body part is a hand, a foot, an arm, a leg, a torso, a phalange, a penis, a nipple, a head, a neck, or a combination thereof.

9. The method of claim 1, wherein the containment reservoir has a volume of from about 0.1 ml to about 20 ml.

10. The method of claim 1, wherein the NORS is introduced in an inactive state.

11. The method of claim 1, wherein the NORS is prepared and activated as it is introduced into the containment reservoir.

12. The method of claim 11, wherein the NORS is introduced as a gel.

13. The method of claim 10, wherein the NORS is activated within the containment reservoir.

14. The method of claim 1, wherein the NORS is formulated to release from about 10 ppm NO to about 5000 ppm NO.

15. The method of claim 1, wherein the NORS is formulated to release NO for a period of from about 30 minutes to about 12 hours.

16. A medicament containment device, comprising:
a housing having an interior wall forming a containment reservoir configured to contain a nitric oxide releasing substance (NORS) therein when the device is attached to a subject; and
an attachment member configured to attach the device to the subject such that nitric oxide is administered to the subject.

17. The device of claim 16, wherein the containment reservoir has a volume of from about 1 ml to about 20 ml.

18. The device of claim 16, wherein the containment reservoir is a hollow channel extending through the housing.

19. The device of claim 16, wherein the containment reservoir includes an inactivated nitric oxide releasing solution (NORS).

20. The device of claim 16, wherein the attachment member comprises a strap configured to wrap about a portion of a body part of the subject.

21. The device of claim 20, wherein the body part is a phalange.

22. The device of claim 21, wherein the strap includes a laterally extending portion configured to wrap around a circumference of the phalange and a tip portion configured to wrap about a tip of the phalange.

23. The device of claim 20, wherein a portion of the strap is configured to wrap about the containment reservoir to cap the containment reservoir.

24. The device of claim 16, wherein the attachment member comprises an adhesive layer configured to couple the containment device to a target treatment situs of the subject.

25. The device of claim 16, further comprising a cap configured for placement over the containment reservoir to direct release of NO from the NORS toward the subject when the device is attached to the subject.
